(19) United States
(12) Patent Application Publication
(10) Pub. No.: US 2007/0181706 A1
(43) Pub. Date: Aug. 9, 2007

(54) SCENT DIFFUSING CLOCK

(76) Inventors: Chia-Lung Yeh, Taipei (TW); Helen Tung, Hsichih, Taipei Hsien (TW)

Correspondence Address: PAI PATENT & TRADEMARK LAW FIRM, 1001 FOURTH AVENUE, SUITE 3200, SEATTLE, WA, 98154, US

(21) Appl. No.: 11/307,397

(22) Filed: Feb. 6, 2006

(51) Int. Cl. A24F 25/00; A61L 9/04; A62C 13/62

(52) U.S. Cl. 239/34; 239/47; 239/302

(57) ABSTRACT

A scent diffusing clock comprises a primary part, a concave part implemented at the middle of said primary part, a recess implemented beside said concave part, a conduit opening being implemented between said concave part and said recess; an air guiding plate placed on a upper end of said primary part, also, a cover being implemented on a upper end of said primary part to hold said primary part and said cover together, a ventilation cover being placed inside said cover, then a plurality of ventilation slots implemented on said ventilation cover; a container plate having a plurality of containers placed thereon, with aromatics of various fragrance being contained in said plurality of containers. Said scent diffusing clock is implemented such that when air is pumped in and guided by said air guiding plate, the fragrance inside said air guiding plate and said container is spread through said ventilation cover.

### BACKGROUND OF THE INVENTION

1. Field of the Invention

The present invention relates to a scent diffusing clock and, more particularly, to a scent diffusing clock combining time-telling function with aromatics of various fragrances to let users know the current time immediately by spreading different fragrances at different times.

2. Description of the Prior Art

Generally a clock uses mechanically operated components to tell time. When a user has set a specific time and that time comes, the clock will ring to inform the user. For example, a cuckoo clock will make a sound resembling that of a cuckoo bird and a man-made cuckoo bird will come out of the cuckoo clock at the preset time. However, the monotonous sound of the cuckoo clock tends to be boring to the users, and the sound made by the cuckoo clock is noisy at night, causing some users to feel uncomfortable.

Therefore, the above-mentioned traditional clocks present several shortcomings to be overcome.

In view of the above-described deficiencies of traditional clocks, after years of constant effort in research, the inventor of this invention has consequently developed and disclosed a scent diffusing clock in the present invention.

### SUMMARY OF THE INVENTION

It is an object of the present invention to provide a scent diffusing clock combining time-telling function with aromatics of various fragrances to let users know the current time immediately by diffusing different fragrances at different times.

It is another object of the present invention to provide a scent diffusing clock having a container plate for containing aromatics of various fragrances such as lavender, rose, jasmine, etc. The fragrance will help people to relax, to feel relieved from stress and to calm nerves, etc., thus providing various functions.

It is still another object of the present invention to provide a scent diffusing clock having a simple structure, which is easy to operate and inexpensive to manufacture.

The scent diffusing clock disclosed in the present invention comprises a primary part, a timing device, an air guiding plate, a cover, a ventilation cover and a container plate. The primary part comprises a concave part implemented at the middle thereof, the concave part being implemented for containing a timing device. Further, a recess is implemented beside the concave part of the primary part, a plurality of openings are evenly placed on the recess, with a protruding part being placed at a lower end of each opening, then a conduit opening is implemented between the concave part and the recess. The conduit opening is connected to a conduit of an air pump for pumping in air and keeping the air circulated. The air guiding plate is placed on an upper end of the timing device of the primary part. The air guiding plate comprises an axle hole implemented at the middle of the air guiding plate for an axle of the timing device to protrude outside the axle hole, one guiding part suitably implemented on each side of the air guiding plate, and a color board implemented on the air guiding plate. The cover is implemented on an upper end of the primary part to hold the components between the primary part and the cover together. A hole is implemented at the middle of the cover, a plurality of open slots are implemented around the hole, a recess is implemented on a perimeter on the plurality of open slots, and a plurality of ventilation holes are implemented and evenly distributed in the recess. The ventilation cover is placed inside the recess of the cover, with a transparent round plate disposed in the middle of the ventilation cover for users to view the pointer in the timing device, then a plurality of ventilation slots are implemented on the ventilation cover. A container plate comprises a plurality of containers thereon, with aromatics of various fragrances contained in the plurality of containers, a film layer placed on a top opening of each container to seal each container, and a handle implemented at the lower end of the container plate. The scent diffusing clock is implemented using components disclosed above. When using it, a user only needs to connect the containers of the container plate with the protruding parts of the openings of the primary part, allowing the protruding parts of the primary part to pierce through the film layer of the container plate, and connect a conduit of an air pump to the conduit opening. When air is pumped in and guided by the air guiding plate, the fragrances inside the air guiding plate and the containers will spread through the ventilation slots of the ventilation cover to let users know the current time immediately from different fragrances at different times.

These features and advantages of the present invention will be fully understood and appreciated from the following detailed description in conjunction with the accompanying Drawings.

### BRIEF DESCRIPTION OF THE DRAWINGS

FIG. 1 illustrates a three-dimensional exploded view of the scent diffusing clock of the present invention;

FIG. 2 illustrates a three-dimensional exploded view of the partially assembled scent diffusing clock;

FIG. 3 illustrates a three-dimensional view of the assembled scent diffusing clock; and

FIGS. 4A, 4B, and 4C illustrate the implementation of the scent diffusing clock.

### DETAILED DESCRIPTION OF THE PREFERRED EMBODIMENT

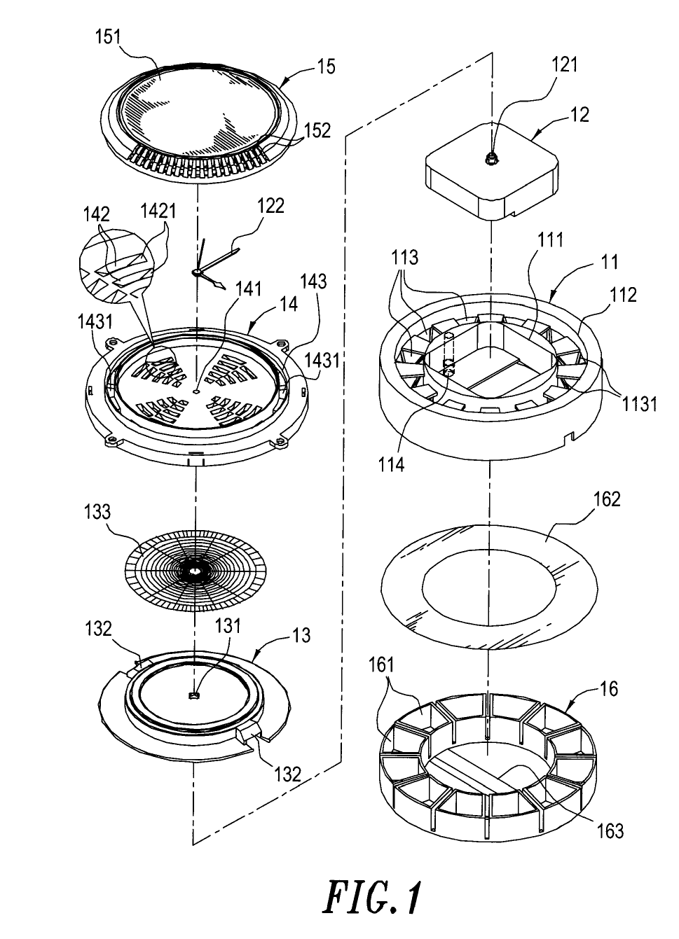

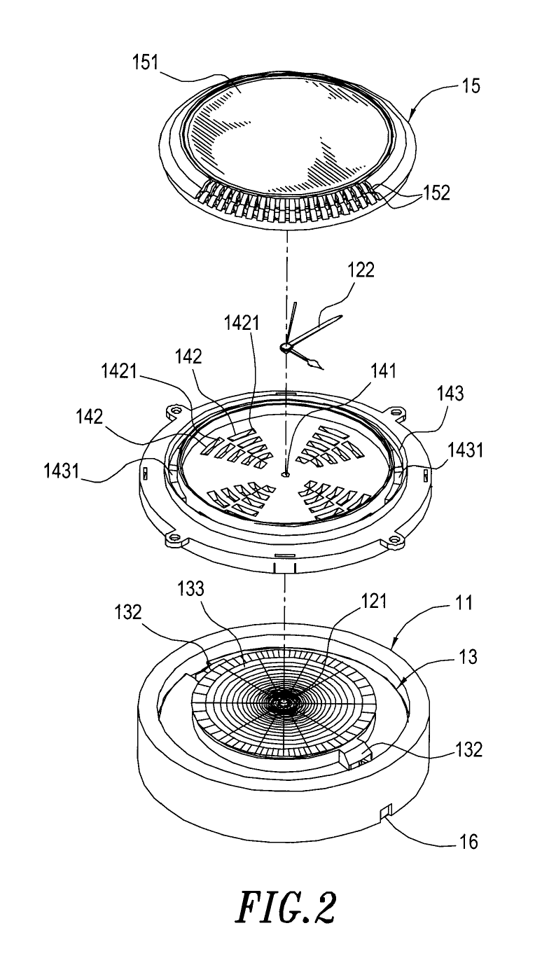

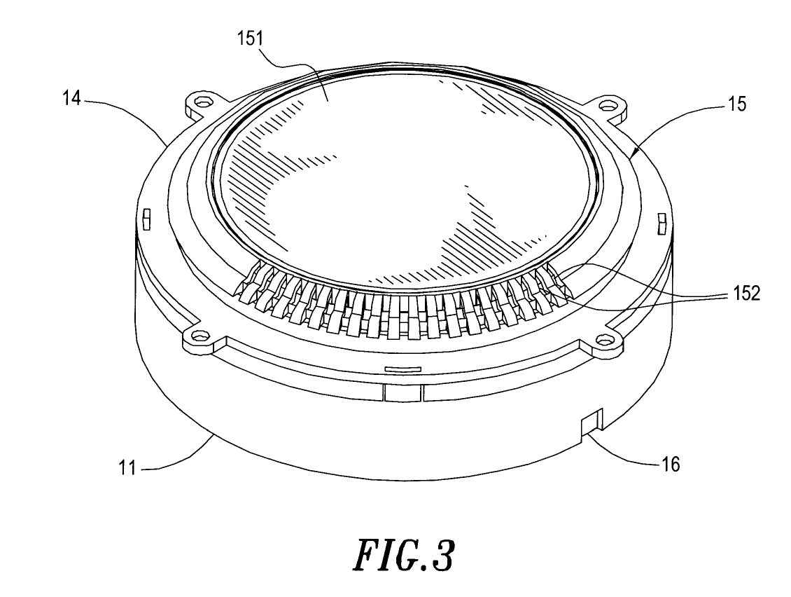

Please refer to FIG. 1 to FIGS. 4A, 4B, and 4C for the scent diffusing clock disclosed in the present invention, which comprises a primary part 11, a timing device 12, an air guiding plate 13, a cover 14, a ventilation cover 15, and a container plate 16.

The primary part 11 comprises a concave part 111 implemented at the middle thereof, a recess 112 implemented beside the concave part 111, a plurality of openings 113 evenly placed on the recess 112 with a protruding part 1131 placed at a lower end of each opening 113, and a conduit opening 114 implemented between the concave part 111 and the recess 112. The conduit opening 114 can be connected to a conduit 2 of an air pump for pumping in air and keeping the air circulated.

The timing device 12 comprises an axle 121 implemented at the middle of the timing device 12 for placing a pointer 122 on the axle 121. The timing device 12 is placed in the concave part 111 of the primary part 11.

The air guiding plate 13 comprises an axle hole 131 implemented at the middle of the air guiding plate 13 for said axle 121 of said timing device to protrude outside the axle hole 131, one guiding part 132 suitably implemented on each side of the air guiding plate 13, and a color board 133 implemented on the air guiding plate 13. The air guiding plate 13 is placed on an upper end of the timing device 12 of the primary part 11. The guiding part 132 of the air guiding plate 13 is used for guiding the direction of the air flowing in the primary part 11, and the color board 133 can have multiple colors applied thereon.

The cover 14 comprises a hole 141 implemented at the middle of the cover 14, a plurality of open slots 142 having inclined planes 1421 implemented around the hole 141, a recess 143 implemented on a perimeter of the plurality of open slots 142, and a plurality of ventilation holes 1431 implemented and evenly distributed in the recess 143. The cover 14 is placed on an upper end of the primary part 11 to hold the components between the primary part 11 and the cover 14 together. The inclined plane 1421 of each open slot 142 can be implemented with mirror material for reflecting light.

The ventilation cover 15 comprises a transparent round plate 151 disposed in the middle of the ventilation cover 15 for users to view the movement of the pointer 122 of the timing device 12, and a plurality of ventilation slots 152 implemented on the ventilation cover 15. The ventilation cover 15 is placed inside the recess 143 of the cover 14.

Finally, the container plate 16 comprises at least two containers 161 placed thereon, with aromatics of various fragrances contained in the containers 161, a film layer 162 placed on a top opening of each container 16 to seal each container 16, and a handle 163 implemented at the lower end of the container plate 16. The container plate 16 contains aromatics of various fragrances such as lavender, rose, jasmine, etc. Each container 161 of the container plate 16 connects with a protruding part 1131 of the primary part 11 correspondingly, and the handle 163 can be used to separate the container plate 16 and the primary part 11 when the primary part 11 and the container plate 16 are connected as a whole.

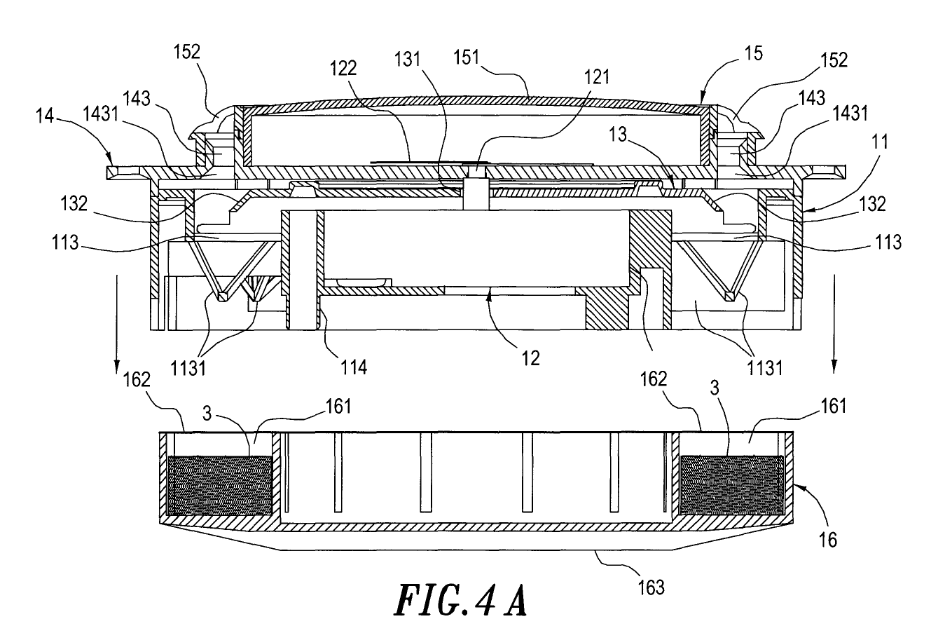

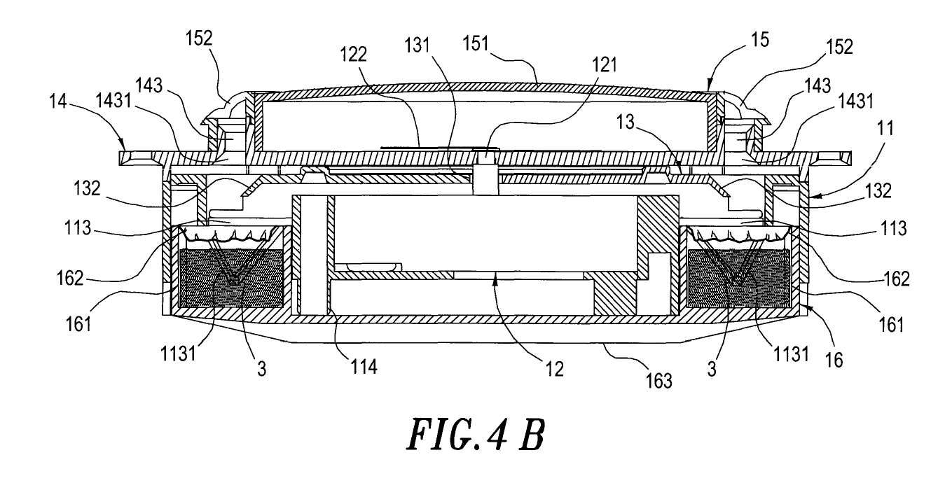

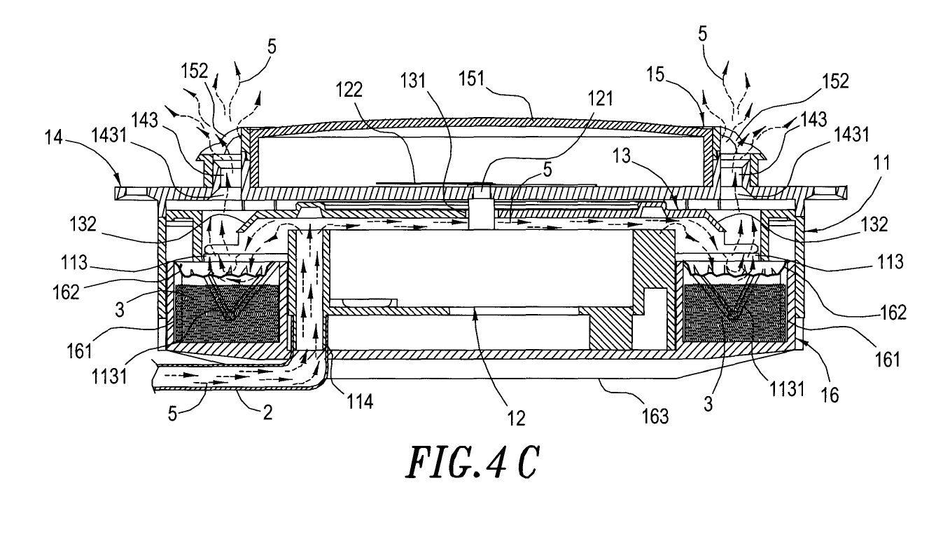

The scent diffusing clock disclosed in the present invention is implemented using components disclosed above, as shown in FIG. 4A. When in use, each container 161 of the container plate 16 is connected correspondingly with a protruding part 1131 of each opening 113 of the primary part 11, then the protruding parts 1131 of the primary part 11 pierce through the film layer 162 of the container plate 16, as shown in FIG. 4B. Furthermore, a conduit 2 of an air pump is connected to the conduit opening 114, when air 5 is pumped in and guided by the guiding part 132 of the air guiding plate 13, the air 5 will flow to the openings 113 of the primary part 11 and eventually come out of the ventilation slots 152 of the ventilation cover 15. When air 5 is circulating, the fragrance inside the containers 161 is diffused outside through the ventilation slots 152 of the ventilation cover 15 to let users know the current time immediately from the different fragrance, as shown FIG. 4C. Furthermore, when all the aromatics 3 in the container plate 16 are evaporated, the handle 163 can be used to separate the container plate 16 and the primary part 11 and the old container plate can be replaced with a new container plate 16 containing new aromatics 3. Besides, the inclined plane 1421 of each open slot 142 of the cover 14 is implemented with mirror material for reflecting the color of the color board 133 projected on the inclined plane 1421 of each open slot 142. Since the air guiding plate 13 is continuously dragged by the axle 121 of the timing device 12, the open slots 142 of the cover 14 change colors as the color board 133 moves around. Therefore, the present invention provides time telling function with different fragrances, and also a clock that can change the color of its surface.

The present invention provides a scent diffusing clock, which, when compared with other traditional clocks, is advantageous in the following aspects:

1. The present invention provides a scent diffusing clock combining time-telling function with aromatics of various fragrances to let users know the current time immediately by diffusing different fragrances at different times.

2. The present invention provides a scent diffusing clock having a container plate for containing aromatics of various fragrances such as lavender, rose, jasmine, etc. The fragrance will help people to relax, to feel relieved from stress and to calm nerves, etc., thus providing various functions.

3. The present invention provides a scent diffusing clock having an inclined plane of each open slot implemented with mirror material for reflecting the color of the color board projected on the inclined plane of each open slot. Since the air guiding plate is continuously dragged by the axle of the timing device, the open slots of the cover change colors as the color board of the air guiding plate moves around. Therefore, the present invention provides time telling function with different fragrances, and also a clock that can change the color of its surface.

4. The present invention provides a scent diffusing clock having a simple structure, which is easy to operate and inexpensive to manufacture.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A scent diffusing clock comprising:
a primary part having a concave part implemented at the middle thereof, a recess implemented beside said concave part, a plurality of openings evenly placed on said recess with a protruding part placed at a lower end of each opening, and a conduit opening implemented between said concave part and said recess;
a timing device having an axle implemented at the middle of said timing device for placing a pointer on said axle;
an air guiding plate having an axle hole implemented at the middle of said air guiding plate, one guiding part implemented on each side of said air guiding plate, and a color board implemented on said air guiding plate;
a cover having a hole being implemented at the middle of said cover, a plurality of open slots having inclined planes implemented around said hole, a recess implemented on a perimeter of said plurality of open slots, and a plurality of ventilation holes implemented and evenly distributed in said recess;
a ventilation cover having a transparent round plate disposed in the middle of said ventilation cover, and a plurality of ventilation slots implemented on said ventilation cover; and
a container plate having at least two containers placed thereon, with aromatics of various fragrances contained in the containers, and a film layer placed on a top opening of each container to seal each container,
wherein said timing device is placed in said concave part of said primary part;
said air guiding plate is placed on an upper end of said timing device;
said axle of said timing device protrudes outside said axle hole of said air guiding plate;
said cover is placed on an upper end of said primary part;
said ventilation cover is placed inside said recess of said cover;
said containers of said container plate are correspondingly connected with said protruding parts of said openings of said primary part; and
said protruding parts of said openings of said primary part pierce through said film layer of said container plate when said primary part is assembled with said container plate.

2. The scent diffusing clock of claim 1, wherein said conduit opening is connected to a conduit of an air pump for pumping in air and keeping the air circulated.

3. The scent diffusing clock of claim 1, wherein said guiding part of said air guiding plate is used for guiding the direction of the air flowing in said primary part.

4. The scent diffusing clock of claim 1, wherein said color board has a plurality of colors applied thereon.

5. The scent diffusing clock of claim 1, wherein said inclined plane of each open slot is implemented with mirror material for reflecting the color of said color board projected on said inclined plane of each open slot, whereby said open slots of said cover change colors as said color board of said air guiding plate is dragged around with said air guiding plate by said axle of said timing device.

6. The scent diffusing clock of claim 1, wherein the aromatics in said container plate have various fragrances.

7. The scent diffusing clock of claim 6, wherein the aromatics in said container plate have fragrances of lavender, rose and jasmine.
